(19) United States
(12) Patent Application Publication
(10) Pub. No.: US 2002/0087158 A1
(43) Pub. Date: Jul. 4, 2002

(54) Combination electrocautery and suction device

(76) Inventor: Terrill McGill, Irvington, NJ (US)

Correspondence Address: Paul E. Schaasfsma, FOLEY & LARDNER, One IBM Plaza, 330 North Wabash Avenue, Suite 3300, Chicago, IL, 60611-3608, US

(21) Appl. No.: 09/753,813

(22) Filed: Jan. 3, 2001

(51) Int. Cl. A61B 18/14

(52) U.S. Cl. 606/45; 606/49; 607/35

(57) ABSTRACT

The present invention provides an electrocautery and suction system 12 that includes an electrocautery device 14, a suction device 16, and a device for controlled extending and retracting of the cauterization device and the suction device 18. The device for controlled extending and retracting 18 includes a suction cartridge 90 and a cautery cartridge 92. The suction cartridge 90 and the cautery cartridge 92 control the extending and retracting of the cauterization device cord 29 and the suction hose 38, respectively.

### FIELD OF THE INVENTION

The present invention relates to medical instruments used to cauterize.

### BACKGROUND OF THE INVENTION

Electrocautery devices are well known in the medical field. Electrocautery devices commonly employ a shaft having a conductive cauterizing element attached to an end. The cauterizing element is connected to an electric source of high frequency electrical energy through appropriate control circuitry. When the high frequency electrical energy flows through the electrical circuitry to the conductive cauterizing element, the current at the cauterization element arcs into the target tissue and travels through the patient to a grounding pad attached to the patient. The heat generated by the current in the tissue causes coagulation of the fluids or cauterization of the tissue, depending on the current level.

One drawback of the use of cauterization, however, is the smoke created during electrocautery. This smoke is generated by the decomposition of the organic material of the cauterized tissue. The amount of smoke generated in such procedures, particularly in less-invasive procedures wherein the procedure is carried out through relatively small ports in the patient, can obscure the view of the surgeon. Additionally, the smoke generated during cauterization at least emits an offensive and irritating odor, and may well be injurious to the health of those present during the procedure.

In order to alleviate the problems caused by this smoke, it is common to provide ventilation to remove the smoke during the cauterization procedure. Such venting comes in several forms, including gas-venting shields, masks that include vacuums sources, and the addition of suction nozzles on the electrocautery device. These devices, however, have proven largely impractical, are relatively expensive, are difficult to clean and sterilize after each use and thus are relatively expensive to use. Thus, many cauterization procedures are performed utilizing separate electrocautery and suction devices.

While the use of separate electrocautery and suction devices is relatively inexpensive and practical, the surgeon performing the procedure is inconvenienced keeping track of these two separate devices. When combined with the use of additional devices in the surgical procedure, the surgeon's task in organizing, tracking and maintaining these devices is compounded.

What would thus be desirable is a device that practically and inexpensively allows for the coagulation of the fluids or cauterization of the tissue. This device should practically and inexpensively provide for the evacuation of the smoke created during electrocautery. This device should further assist the surgeon performing the procedure in organizing, tracking and maintaining surgical devices. This device should enable the surgeon to keep their eye on the surgical site, while releasing the instrument but knowing that the instrument is not out of their reach.

### SUMMARY OF THE INVENTION

The present invention provides a device that practically and inexpensively allows for the coagulation of the fluids or cauterization of the tissue. The present invention practically and inexpensively provides for the evacuation of the smoke created during electrocautery. The present invention further assist the surgeon performing the procedure in organizing, tracking and maintaining surgical devices. The present invention enables the surgeon to keep their eye on the surgical site, while releasing the instrument but knowing that the instrument has not fallen out of their reach.

The present invention provides an electrocautery and suction system that includes an electrocautery device, a suction device, and a device for controlled extending and retracting of the cauterization device and the suction device. The device for controlled extending and retracting includes a suction cartridge and a cautery cartridge. The suction cartridge and the cautery cartridge control the extending and retracting of the cauterization device cord and the suction hose, respectively.

### BRIEF DESCRIPTION OF THE DRAWINGS

FIG. 1 is a schematic of an electrocautery and suction system in accordance with the principles of the present invention.

FIG. 2 is a perspective view of a device for controlling, extending and retracting in accordance with the principles of the present invention.

FIG. 3 is a perspective view of the suction and cautery cartridges of FIG. 2.

### DETAILED DESCRIPTION OF THE PREFERRED EMBODIMENTS

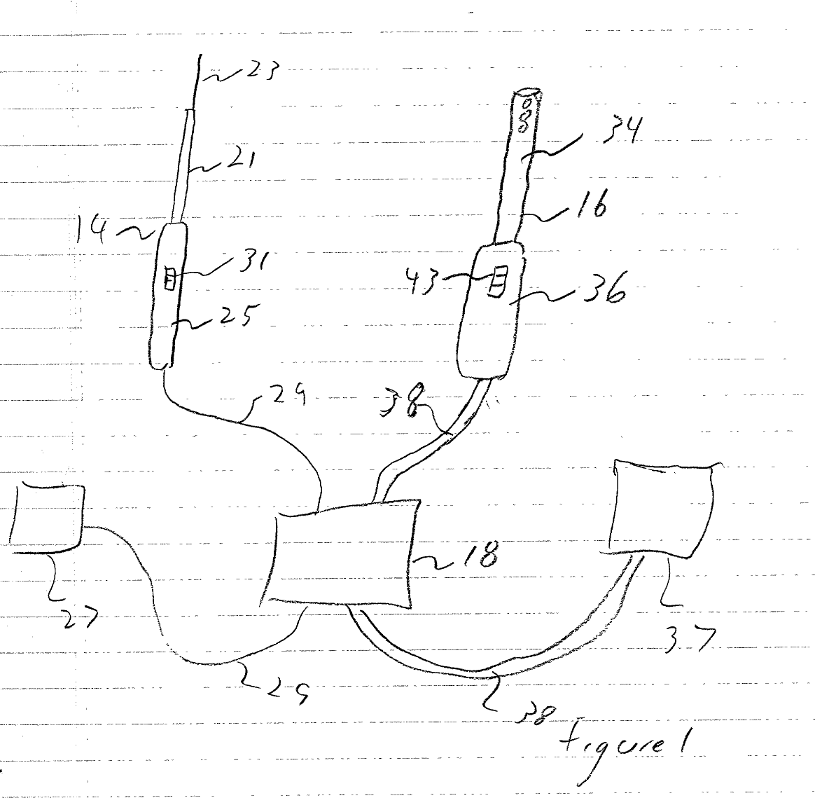

Referring first to FIG. 1, a schematic of an electrocautery and suction system in accordance with the principles of the present invention is generally designated at 12. The system includes an electrocautery device 14, a suction device 16, and a device for controlled extending and retracting 18. The electrocautery device 14 generally includes an insulated shaft 21 having an electrode 23 extending from one end thereof. At the opposite end, a handle 25 is provided.

The electrocautery device 14 is coupled to a source of electrical energy 27 by means of a power cord 29. The power cord 29 provides electrical energy to the electrode 23. The handle 25 can include a switch 31 for selectively actuating the electrocautery device 14.

The suction device 16 generally includes an insulated tube 34 defining an open lumen through which the fluids are evacuated. At the end of the suction device 16 opposite the open lumen, a handle 36 is provided. The suction device 16 is coupled to a source of vacuum 37 by means of a hose 38. The handle 36 can include a switch 43 for selectively actuating the suction device 16.

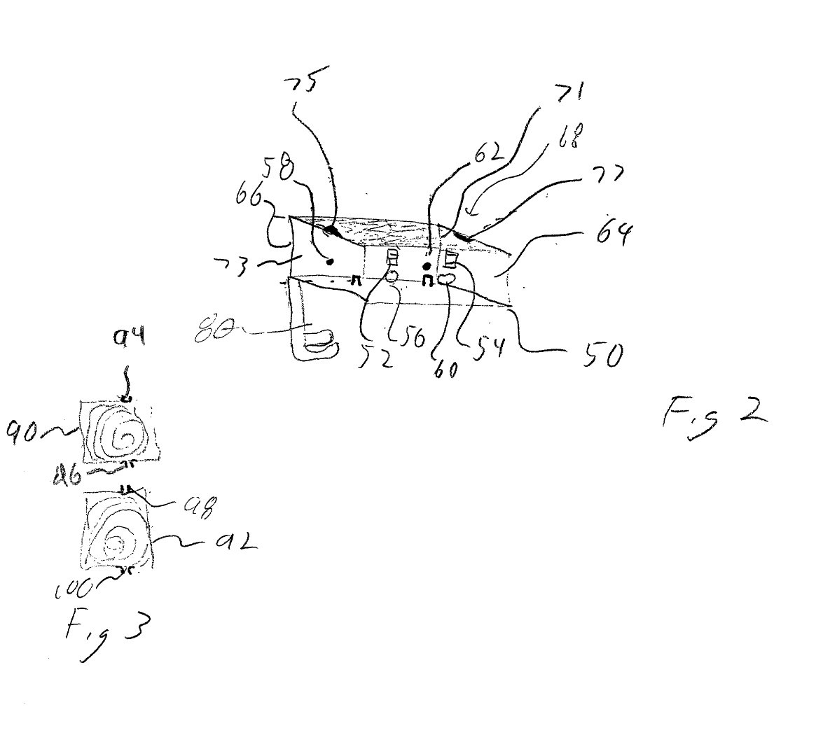

Referring to FIG. 2, a perspective view of the device for controlled extending and retracting 18 in accordance with the principles of the present invention is seen. The device for controlling generally includes a housing 50. In the preferred embodiment, the housing 50 can consist of a stainless steel box. The housing defines a pair of switches 52, 54. The housing 50 further defines four apertures 56, 58, 60, 62, with one pair of apertures 56, 60 preferably located on the “front” side 64 of the housing 50 and a second pair 58, 62 preferably located on the “back” side 66 of the housing 50.

The housing 50 is further divided into an upper portion 71 and a lower portion 73. The upper portion 71 and the lower portion 73 are removable secured to each other, preferably by means of a pair of latches 75, 77. The latches 75, 77 are positioned opposite one to another. Each latch 75, 77 will be securely fastened while surgery is being performed. These latches 75, 77, once released, enable the upper portion 71 of the housing 50 to be removed. Once removed, the housing 50 inner components could be exposed to allow for three features:

(1) The insertion of a cartridge for a tube or cautery change.

(2) The cleansing of the device's inner components.

(3) Storage of the suction tube and cauterization cords.

The pair of apertures 56, 60 defined on the “front” side 64 of the housing 50 enable the cauterization device 14 and suction device 16 to be made ready for use by the surgeon or the operating room staff. The pair of apertures 58, 62 defined on the “back” side 66 of the housing 50 enable the cauterization device cord 29 and the suction device hose 38 to be attached to their respective sources 27, 37. Finally, a bracket 80 is provided to secure the device for controlling, extending and retracting 18 to, for example, a bed.

Referring now to FIG. 3, a perspective view of the suction cartridge 90 and cautery cartridge 92 of FIG. 2 is seen. The suction cartridge 92 provides for the extension and retraction of the suction hose 38 while the cautery cartridge 92 provides for the extension and retracting of the cautery cord 29. Thus, the suction cartridge 90 includes an opening 96 for the suction hose 38 and a second opening 98 for the insulated tube 34. Likewise, the cautery cartridge 92 includes an opening for the cautery cord 29 and a second opening for the electrode 23. The suction cartridge 90 and the cautery cartridge 92 can consist of a biased element such as a spring to provide resistance to extension and assistance to retraction, as known.

The switches 52, 54 control the extending and retracting of the suction hose 38 and the cautery cord 29. One switch is positioned above the aperture 56 defined on the “front” side of the housing 50 and enables the cauterization device 14 to be made ready for use by the surgeon or the operating room staff. The other switch 54 is positioned above the aperture 60 defined on the “front” side of the housing 50 and enables the suction device 16 to be made ready for use by the surgeon or the operating room staff.

In accordance with the principles of the present invention, when the surgeon is at a surgical site and is ready to use the cauterization device 14 or the suction device 16, the cauterization device 14 or the suction device 16 can be detracted from the device for controlled extending and retracting 18. Upon the surgeon's release of the cauterization device 14 or the suction device 16, the cauterization device 14 or the suction device 16 will retract back to its original position within the device for controlled extending and retracting 18. This retraction effect will enable the surgeon to keep their eye on the surgical site, be able to release an instrument and know that the instrument has not fallen out of their reach.

In the “off” position, the switches 52, 54 allow the respective instrument 14, 16 to be pulled from the original positions and extend to the surgical site. At the surgeon's request, either switch 52, 54 may be placed in the “on” position. In the “on” position, the suction hose 38 or the cautery cord 29 is restricted, confining that instrument 14, 16 to a specific surgical site. Thus, the present invention allows a surgeon to use the cauterization device 14 individually, the suction device 16 individually, or both the cauterization device 14 and the suction device 16 together at one time.

It should be understood that various changes and modifications to the preferred embodiment described herein would be apparent to those skilled in the art. Such changes and modifications can be made without departing from the spirit and scope of the present invention and without demising its attendant advantages. It is therefore intended that such changes and modifications be covered by the appended claims.

What is claimed is:

1. An electrocautery and suction system comprising:
an electrocautery device;
a suction device; and
a device for controlled extending and retracting of the cauterization device and the suction device.

2. The electrocautery and suction system of claim 1 further wherein the electrocautery device generally includes an insulated shaft having an electrode extending from one end thereof.

3. The electrocautery and suction system of claim 2 further wherein the electrocautery device generally includes handle on the end opposite the electrode.

4. The electrocautery and suction system of claim 3 further wherein the handle includes a switch for selectively actuating the electrocautery device.

5. The electrocautery and suction system of claim 2 further wherein he electrocautery device is coupled to a source of electrical energy.

6. The electrocautery and suction system of claim 5 further wherein the electrocautery device is coupled to a source of electrical energy by a power cord.

7. The electrocautery and suction system of claim 1 further wherein the suction device includes an insulated tube defining an open lumen through which fluids are evacuated.

8. The electrocautery and suction system of claim 7 further wherein the suction device includes a handle on the end opposite the tube.

9. The electrocautery and suction system of claim 8 further wherein handle includes a switch for selectively actuating the suction device.

10. The electrocautery and suction system of claim 7 further wherein the suction device is coupled to a source of vacuum.

11. The electrocautery and suction system of claim 10 further wherein the suction device is coupled to a source of vacuum by a hose.

12. The electrocautery and suction system of claim 1 further wherein the device for controlling, extending and retracting includes a pair of switches, with each switch respectively controlling the extending and retracting of the cauterization device cord and the suction hose.

13. The electrocautery and suction system of claim 1 further wherein the device for controlling the extending and retracting is divided into a lower portion and a removably secured upper portion.

14. The electrocautery and suction system of claim 1 further wherein the device for controlling the extending and retracting includes a suction cartridge and a cautery cartridge to provide for the extension and retraction of the suction hose and the cautery cord.

15. The electrocautery and suction system of claim 14 further wherein the cartridges consist of a biased element to provide resistance to extension and assistance to retraction.

16. The electrocautery and suction system of claim 15 further wherein the biased element is a spring.

17. A method for cauterizing and suctioning comprising:
extending from a device a cauterization device;
extending from a device a suction device;
retracting from the device the cauterization device; and
retracting from the device the suction device.

18. The method for cauterizing and suctioning of claim 17 further including cauterizing with the cauterization device.

19. The method for cauterizing and suction claim 18 further wherein the cauterizing includes providing electrical energy.

20. The method for cauterizing and suctioning of claim 18 further including selectively actuating the electrocautery device.

21. The method for cauterizing and suctioning of claim 17 further including suctioning with the suction device.

22. The method for cauterizing and suctioning of claim 18 further including selectively actuating the suction device.

23. The method for cauterizing and suctioning of claim 17 further including selectively actuating the extending and retracting of the cauterization device and the suction device.
